SEQUENCE LISTING (2 sequences)

SEQ ID NO: 1 - DNA, 72 bp, Artificial Sequence
  forward primer
atggtgccgtccattattatcgcggatgccggaagttaacgaagctattcttgtgtaggctggagctgcttc

SEQ ID NO: 2 - DNA, 70 bp, Artificial Sequence
  reverse primer
gataaccctccgcagcgtcacctttacgcactttgtgatagccggtaatcatgggaattagccatggtcc

(19) United States
(12) Patent Application Publication
(10) Pub. No.: US 2022/0213520 A1
(43) Pub. Date: Jul. 7, 2022

(54) COLANIC ACID PRODUCTION USING MUTANT E. COLI

(71) Applicant: Korea University Research and Business Foundation, Seoul (KR)

(72) Inventors: Kyoung Heon KIM, Seoul (KR); Yong-Su JIN, Champaign, IL (US); Eun Ju YUN, Seoul (KR); In Jung KIM, Seoul (KR); Hyeong Min HAN, Seoul (KR); Sora YU, Namyangju-si (KR)

(73) Assignee: Korea University Research and Business Foundation, Seoul (KR)

(21) Appl. No.: 17/606,263

(22) PCT Filed: Apr. 24, 2020
(86) PCT No.: PCT/KR2020/005439

§ 371 Date: Dec. 14, 2021

(30) Foreign Application Priority Data
Apr. 26, 2019 (KR) 10-2019-0049226

(51) Int. Cl. C12P 19/04; C12N 1/20; C12N 9/10

(52) U.S. Cl. CPC C12P 19/04; C12N 9/1048; C12N 1/20

(57) ABSTRACT

The present invention relates to a medium composition for culturing a strain for mass production of colanic acid and a method of mass producing colanic acid using the same. Ingredients for the culture medium of the present invention and their concentrations may be optimized using a statistical method, and used to greatly increase the production amount of colanic acid.

### TECHNICAL FIELD

The present invention relates to a method of producing colanic acid using mutant E. coli.

### BACKGROUND ART

Colanic acid is one of the extracellular polysaccharides, which has a negative charge and a molecular weight of 3.4 kDa, and is known to be produced by various bacteria belonging to the family Enterobacteriaceae forming a biofilm and growing. It is estimated that colanic acid plays an important role in the formation of the three-dimensional structure of the biofilm, and imparts resistance to phage infection to bacteria in the biofilm, resistance to environmental factors such as osmotic pressure, dehydration, low temperature and oxidative stress, and resistance to an antibiotic. The structure of colanic acid is a structure with repeated six sugars including, for example, two fucoses, two galactoses, one glucose and one glucuronic acid. In addition, colanic acid has acetic acid and pyruvic acid as residues. Fucose, which is one of the monomeric sugars, is a rare sugar that cannot be easily obtained, but is widely used in food, medical and cosmetic fields due to several physiologically active functions. As a food material, colanic acid is used as a coagulant, a film-forming agent, a gel-forming agent or an emulsion stabilizer due to high moisture binding strength, and also used as a diet sugar due to having low calories. For pharmaceuticals, colanic acid is used as an anti-inflammatory agent, an anticancer agent and an adjuvant, and widely used as a cosmetic material due to whitening, moisturizing, dermal cell regeneration-promoting and anti-aging effects. However, despite these uses, a method of obtaining colanic acid is difficult and has a low yield, so it is very expensive. Colanic acid aiming at the maximum production, however, has fucose as a monomer, which accounts for about ⅓ of the total mass, and is able to be used in fucose production if colanic acid is mass-produced. In addition, according to a recent research result, various physiological activities of colanic acid itself have been revealed, and the utility value thereof is increasing.

However, there are no studies on optimizing media for mass production of colanic acid using microorganisms. In fact, when looking at studies for producing different types of extracellular polysaccharides, taking into account the fact that the production amount of extracellular polysaccharides is greatly changed according to changes in medium ingredients, it can be seen that a study on medium optimization is essential.

### RELATED ART DOCUMENT

### Non-Patent Document

Front. Microbiol. 6:496 (2015), Bacterial exopolysaccharides: biosynthesis pathways and engineering strategies.

### DISCLOSURE

### Technical Problem

The inventors developed an optimal strain for producing colanic acid based on a previous study in that an incomplete lipopolysaccharide is formed by removing the waaF gene among genes involved in the formation of lipopolysaccharides constituting a cell membrane, and as a result, when the corresponding cells are exposed to external stress, colanic acid is produced by a defense mechanism against the stress, and a method of mass producing colanic acid was completed through culture medium optimization.

Therefore, the present invention is directed to providing a method of mass producing colanic acid through medium optimization using a mutant E. coli JM109 strain from which the waaF gene is removed.

### Technical Solution

The present invention relates to a method of producing colanic acid, which includes:

preparing a mutant E. coli JM109 strain by removing the waaF gene from a E. coli JM109 strain; and

culturing the prepared mutant E. coli JM109 strain in a fermentation medium.

To remove the waaF gene from the E. coli JM109 strain corresponding to a known strain, known genetic engineering technology for removing a specific gene may be used without limitation. In an exemplary embodiment of the present invention, a waaF gene-removed mutant E. coli JM109 strain was prepared using λ-red recombination technology shown in FIG. 1.

In an exemplary embodiment of the present invention, when glucose is used as a carbon source and tryptone is used as a nitrogen source by confirming colanic acid production yields of the strain for various carbon and nitrogen sources to optimize the composition and concentration of a fermentation medium colanic acid, excellent productivity was confirmed.

Accordingly, the fermentation medium may include glucose, tryptone and sodium phosphate (Na₂HPO₄), and further include sodium chloride (NaCl), magnesium sulfate (MgSO₄), calcium chloride (CaCl₂) and potassium phosphate (KH₂PO₄).

The fermentation medium may include 10 to 30 g/l of glucose, 7 to 15 g/l of sodium phosphate, 1 to 5 g/l of potassium phosphate, 0.1 to 1 g/l of sodium chloride, 1 to 5 g/l of tryptone, 0.1 to 0.5 g/l of magnesium sulfate, and 0.005 to 0.02 g/l of calcium chloride.

The inventors selected variables having the greatest effect on the production amount of colanic acid among various ingredients contained in a medium mixture, minimized the enormous number of experimental conditions and thus simply and effectively selected the optimal condition for the medium using a fractional factorial design, a steepest ascent method and response surface methodology in order to optimize a strain fermentation medium.

Therefore, in an exemplary embodiment of the present invention, the optimized fermentation medium may include 20 g/l of glucose, 10.62 g/l of sodium phosphate, 3.00 g/l of potassium phosphate, 0.5 g/l of sodium chloride, 2.63 g/l of tryptone, 0.24 g/l of magnesium sulfate and 0.011 g/l of calcium chloride.

In the fermentation medium, the culture of a mutant E. coli strain may be performed at 20 to 30° C., and specifically, at 25° C.

Prior to the culture of the mutant E. coli strain in a fermentation medium, preculture in a LB medium may be further included.

The LB medium contains agar, and the pre-culture may be performed at 30 to 40° C.

### Advantageous Effects

A method of producing colanic acid according to the present invention is for optimizing a strain and a culture medium to be suitable for colanic acid production, and the production of colanic acid is significantly increased compared to that before optimization.

### DESCRIPTION OF DRAWINGS

FIG. 1 is a schematic diagram illustrating a λ-red mediated recombination method for removing the waaF gene from an E. coli JM109 strain.

FIG. 2 shows the comparison in (A) the production of colanic acid and (B) cell growth according to the type and concentration of a carbon source present in a medium for culturing a waaF gene-deleted E. coli JM109 strain.

FIG. 3 shows the comparison in the production of colanic acid and (B) cell growth according to the concentration of glucose, which is a carbon source present in a medium for culturing a waaF gene-deleted E. coli JM109 strain.

FIG. 4 shows the comparison in (A) the production of colanic acid and (B) cell growth according to the type and concentration of a nitrogen source present in a medium for culturing a waaF gene-deleted E. coli JM109 strain.

FIG. 5 is a three-dimensional response surface plot based on central composite design of the production amount of colanic acid according to the concentrations of tryptone and sodium phosphate, dibasic (Na₂HPO₄).

FIG. 6 shows the result of culturing a waaF gene-deleted E. coli JM109 strain under optimal medium conditions (20.00 g/l of glucose, 2.63 g/l of tryptone, 10.62 g/l of Na₂HPO₄, 3.00 g/l of KH₂PO₄, 0.50 g/l of NaCl, 0.24 g/l of MgSO₄, and 0.011 g/l of CaCl₂).

### MODES OF THE INVENTION

Hereinafter, the present invention will be described in detail with reference to the following examples. The following examples are merely provided to exemplify the present invention, and the contents of the present invention are not limited to the following examples.

### [Example 1] Gene Removal

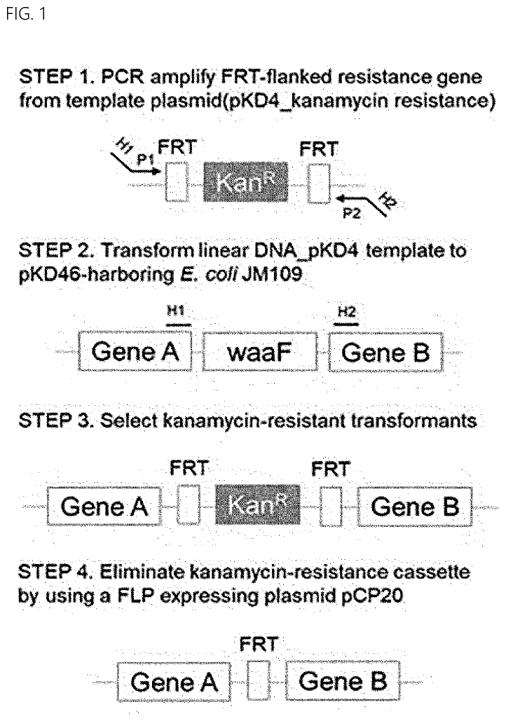

A strain used in the present invention is E. coli JM109 ΔwaaF, and the waaF gene was removed from a general E. coli JM109 strain. A strategy used for gene removal is λ-red recombination technology, and is schematically illustrated in FIG. 1. The detailed process is as follows:

1) A sequence fragment including a kanamycin-resistant gene between flippase recognition targets (FRTs), and upstream and downstream genes having homology with flanking regions of waaF was amplified using pKD4 plasmids by PCR.

The primer set used for the amplification of the sequence fragment is as follows:

Forward primer:
(SEQ ID NO: 1)
5′-ATGGTGCCGTCCATTATTATCGCGGATGCCGGAAGTTAACGAAG
CTATTCTTGTGTAGGCTGG AGCTGCTTC-3′
and
Reverse primer:
(SEQ ID NO: 2)
5′-GATAACCCTCCGCAGCGTCACC TTTACGCACTTTGTGATAGCC
GGTAATCATGGGAATTAGCCATGGTCC-3′.

The underlined sequence areas of the primers indicate the homologous recombination areas of waaF.

2) A pKD46 plasmid was inserted into E. coli JM109.

3) A linear DNA template amplified in 1) was inserted into the E. coli JM109 into which the pKD46 plasmid was inserted, and then the pKD46 plasmid was expressed.

4) A strain from which only the waaF gene was removed from the conventional E. coli JM109 was completed by insertion and expression of a pCP20 plasmid expressing a flippase recombination protein.

### [Example 2] Fermentation Conditions

As a basic medium used for medium optimization, a M9 minimal medium, which is a medium that can easily show the influence of each ingredient and has minimal effect on analysis was selected, and the composition of the minimal medium was partially changed, and the concentration range of the medium ingredients was varied for use. Particularly, before optimization, the composition of the M9 minimal medium is as follows: 10.00-30.00 g/l of glucose, 1.00-2.00 g/l of tryptone, 3.00-10.00 g/l of sodium phosphate (Na₂HPO₄), 1.50-4.50 g/l of potassium phosphate (KH₂PO₄), 0.12-0.36 g/l of magnesium sulfate (MgSO₄), 0.005-0.017 g/l of calcium chloride (CaCl₂) and 0.50 g/l of sodium chloride (NaCl). As a result of investigation, it was found that when a temperature was low, a large amount of colanic acid is produced, so fermentation was performed at 25° C. In addition, as detailed conditions, the cultivation was carried out in a 250 ml Erlenmeyer flask containing a 50 ml medium by shaking culture at 200 rpm, and then after 24 hours, the degree of bacterial growth and the production amount of colanic acid were measured.

### [Example 3] Quantification of Colanic Acid

The quantification of colanic acid was carried out in the manner of specifically quantifying glucuronic acid, which is one of the monomers. After the recovery of the cultured medium, and then the medium was reacted at 90 to 95° C. for 10 minutes to inactivate a protein. The resulting solution was then centrifuged at 4° C. and 10,000×g for 30 minutes, thus bacterial cells were separated in the form of a pellet, and colanic acid was present in a supernatant. After recovery of only the supernatant, ethanol was added at a volume corresponding to three times the volume of the supernatant to precipitate colanic acid. Again, the precipitate was recovered through centrifugation at 4° C. and 10,000×g for 30 minutes, dissolved in distilled water and used for the quantification of glucuronic acid. The quantification of glucuronic acid was carried out as follows:

1) 5 ml of a 12.5 mM sodium tetraborate-sulfuric acid solution was added to 1 ml of a sample, and reacted at 100° C. for 5 minutes.

2) After sufficiently cooling, 100 μl of a solution in which hydroxydiphenyl was dissolved in a 0.5% (w/v) sodium hydroxide aqueous solution at 1.5 g/l was added and sufficiently mixed.

3) A glucuronic acid concentration of the corresponding sample was calculated by substituting the absorbance of the solution measured at 526 nm into a standard graph.

4) The standard graph was plotted with a glucuronic acid standard solution.

### [Example 4] Selection of Optimal Carbon Source

The degree of bacterial growth and the production amount of colanic acid were measured by changing only the type and concentration of a carbon source while leaving other ingredients of the MO minimal medium as they are. As carbon sources, a total of six sources such as glucose, sucrose, glycerol, xylose, molasses and a malt extract were used. Specifically, in the fermentation medium containing 6.78 g/l of Na₂HPO₄, 3.00 g/l of KH₂PO₄, 0.50 g/l of NaCl, 1.00 g/l of NH₄Cl, 0.24 g/l of MgSO₄ and 0.011 g/l of CaCl₂,

the carbon source was added at a concentration of 5, 10, 15 or 20 g/l and the production amount of colanic acid was measured.

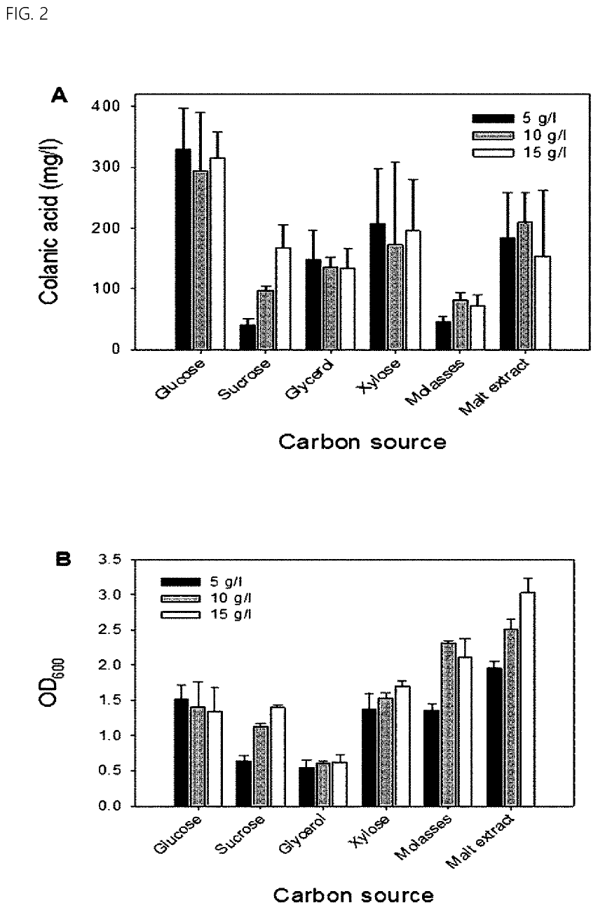

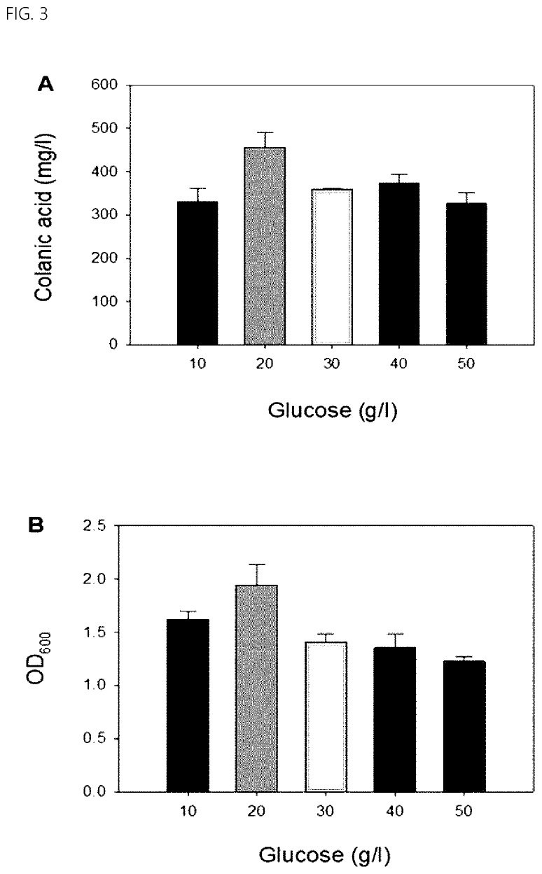

As shown in FIG. 2, when glucose was used as a carbon source, since colanic acid was produced at the highest level and the result of bacterial growth was not bad, glucose was selected as the optimal carbon source. However, since it was difficult to confirm the difference according to a concentration, a reinforcement experiment was carried out by concentration. Referring to FIG. 3, when the concentration of glucose was 20 g/l, the production amount of colanic acid and the bacterial growth were the highest, and thus the subsequent experiment was carried out with 20 g/l of glucose as a carbon source.

### [Example 5] Selection of Optimal Nitrogen Source

As known from the previous experiment, 20 g/l of glucose was selected as a carbon source, and the bacterial growth and the production amount of colanic acid were measured with various types and concentrations of nitrogen sources while leaving other factors as they are. A total of 7 nitrogen sources, which include peptone, tryptone, a yeast extract, urea, and a corn concentrate as organic nitrogen sources, and ammonium sulfate and ammonium chloride as inorganic nitrogen sources, were used. Specifically, the production amount of colanic acid was measured by adding 0.5, 1 or 1.5 g/l of the nitrogen source to a fermentation medium including 20.00 g/l of glucose, 6.78 g/l of Na₂HPO₄, 3.00 g/l of KH₂PO₄, 0.50 g/l of NaCl, 0.24 g/l of MgSO₄, and 0.011 g/l of CaCl₂.

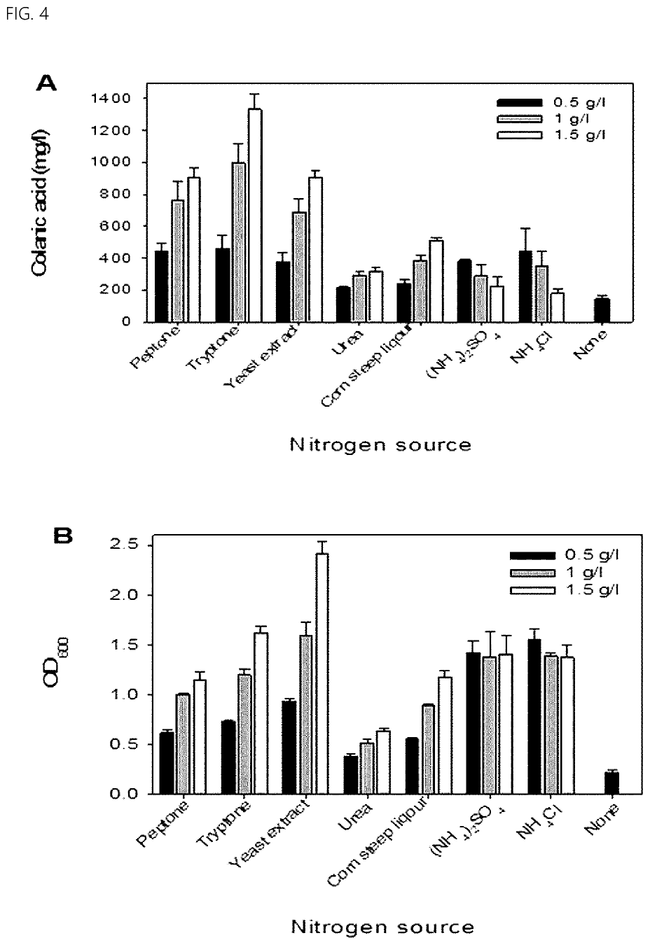

As shown in FIG. 4, when tryptone was provided, the largest production amount of colanic acid was shown. In terms of bacterial growth, the tryptone showed the best result following a yeast extract. Therefore, tryptone was selected as a nitrogen source, and the subsequent experiment was carried out with 1.5 g/l tryptone.

As a result, according to Examples 4 and 5, glucose and tryptone were used as a carbon source and a nitrogen source, respectively, and the final composition of the M9 minimal medium included glucose, sodium phosphate, potassium phosphate, sodium chloride, tryptone, magnesium sulfate and calcium chloride.

### [Example 6] Statistical Method for Optimizing Culture Medium for Colanic Acid Synthesis

<6-1> Fractional Factorial Design (FFD)

As the first step for optimizing the concentrations of medium ingredients in earnest, FFD is an experiment for examining how much each component affects the production amount of colanic acid.

To screen the most important ingredients in a culture medium that affects colanic acid production, FFD was made using Minitab 18.1 (Minitab, State College, Pa., USA). FFD formed 18 experimental mixtures consisting of six independent variables with three levels (−1, 0 and 1) (Tables 1 and 2). In FFD, the amounts (g/l) of glucose (X1), tryptone (X2), Na₂HPO₄ (X3), KH₂PO₄ (X4), MgSO₄ (X5) and CaCl₂ (X6) were used as independent variables, and the amount (mg/1) of colanic acid produced by E. coli JM109 ΔwaaF (Y1) and a cell density (OD600; Y2) was used as dependent variables. The coded value of the dependent variables was obtained by the following equation:

$${x}_{i} = \frac{{X}_{i} - {X}_{0}}{Δ {X}_{i}}$$

Here, xᵢ is a level (coded value) of a medium ingredient, Xᵢ is an actual value of a medium ingredient at the level xᵢ, X₀ is an actual value of a medium ingredient at the baseline, and ΔXᵢ is a step change value. All experiments were performed in triplicate.

Based on the experimental result of FFD, regression analysis was performed to identify a component having a significant effect on colanic acid production. It simply means that when Xᵢ has a high absolute value of a coefficient estimate, Xᵢ has an important effect on colanic acid production. When Xᵢ is a negative coefficient estimate, it means that Xᵢ has a negative effect on colanic acid production, and when Xᵢ is a positive coefficient estimate, there is a positive effect.

Experimental concentrations were determined based on the M9 minimal medium and previous experiments, and are summarized in Table 1. FFD was designed with the determined concentrations, and after the experiment was carried out according to the design, the degree of bacterial growth and the production amount of colanic acid were measured and summarized in Table 2. To obtain an exact result, regression analysis was performed, and the regression analysis result is shown in Table 3. As a result of regression analysis, it was confirmed that X2 and X3, that is, tryptone and Na₂HPO₄ have a great effect on the production amount of colanic acid, and as a concentration increases in the determined concentration range, it was found that the production amount of colanic acid increases. Therefore, as a subsequent experiment, an experiment of optimizing concentrations of these two ingredients was carried out.

TABLE 1
Setting of FFD concentration range

| Independent variable | | Level^{a} | | |
|---|---|---|---|---|
| (g/l) | Variable | −1 | 0 | +1 |
| X₁ | Glucose | 10 | 20 | 30 |
| X₂ | Tryptone | 1.0 | 1.5 | 2.0 |
| X₃ | Na₂HPO₄ | 3.78 | 6.78 | 9.78 |
| X₄ | KH₂PO₄ | 1.5 | 3.0 | 4.5 |
| X₅ | MgSO₄ | 0.12 | 0.24 | 0.36 |
| X₆ | CaCl₂ | 0.005 | 0.011 | 0.017 |

^{a}x₁ = (X₁ − 20)/10; x₂ = (X₂ − 1.5)/0.5; x₃ = (X₃ − 6.78)/3; x₄ = (X₄ − 3)/1.5; x₅ = (X₅ − 0.24)/0/12; x₆ = (X₆ − 0.011)/0.006

TABLE 2
Experiment design by FFD and its result

| Run | x₁ | x₂ | x₃ | x₄ | x₅ | x₆ | Y₁ (CA; mg/l)^{a} | Y₂ (OD₆₀₀)^{a} |
|---|---|---|---|---|---|---|---|---|
| 1 | −1 | 1 | 1 | −1 | −1 | −1 | 1289.8 ± 20.1 | 2.02 ± 0.05 |
| 2 | 1 | 1 | −1 | −1 | −1 | 1 | 822.1 ± 71.6 | 1.58 ± 0.05 |
| 3 | −1 | 1 | −1 | −1 | 1 | 1 | 752.4 ± 30.4 | 1.79 ± 0.04 |
| 4 | 1 | 1 | −1 | 1 | −1 | −1 | 817.3 ± 17.9 | 1.66 ± 0.08 |
| 5 | 0 | 0 | 0 | 0 | 0 | 0 | 1201.7 ± 72.8 | 1.71 ± 0.06 |
| 6 | −1 | −1 | 1 | −1 | 1 | 1 | 1075.3 ± 33.9 | 1.18 ± 0.03 |
| 7 | −1 | −1 | −1 | −1 | −1 | −1 | 671.5 ± 51.6 | 1.22 ± 0.01 |
| 8 | 1 | −1 | −1 | −1 | 1 | −1 | 690.6 ± 20.7 | 1.07 ± 0.09 |
| 9 | −1 | 1 | 1 | 1 | −1 | 1 | 1287.0 ± 13.1 | 2.43 ± 0.03 |
| 10 | −1 | −1 | −1 | 1 | −1 | 1 | 224.0 ± 11.7 | 1.48 ± 0.02 |
| 11 | 1 | 1 | 1 | 1 | 1 | 1 | 1447.3 ± 88.8 | 2.33 ± 0.09 |
| 12 | 1 | −1 | 1 | −1 | −1 | 1 | 1075.3 ± 24.4 | 1.24 ± 0.07 |
| 13 | 1 | −1 | 1 | 1 | −1 | −1 | 849.1 ± 15.8 | 1.27 ± 0.01 |
| 14 | 1 | 1 | 1 | −1 | 1 | −1 | 1210.6 ± 45.7 | 2.09 ± 0.08 |
| 15 | 1 | −1 | −1 | 1 | 1 | 1 | 676.3 ± 32.3 | 1.31 ± 0.05 |
| 16 | 0 | 0 | 0 | 0 | 0 | 0 | 1356.3 ± 49.8 | 1.66 ± 0.01 |
| 17 | −1 | −1 | 1 | 1 | 1 | −1 | 770.8 ± 18.8 | 1.41 ± 0.08 |
| 18 | −1 | 1 | −1 | 1 | 1 | −1 | 954.8 ± 73.6 | 1.76 ± 0.08 |

^{a}Data were expressed as means ± standard deviations of triplicate experiments

TABLE 3
Regression analysis for FFD result

| Source | Coefficient estimate | Mean square | F-value | p-value^{a} |
|---|---|---|---|---|
| Model | | 117087 | 13.09 | 0.028 |
| Intercept | 913.4 | | | 0.000 |
| X₁ | 35.2 | 19814 | 2.22 | 0.233 |
| X₂ | 159.3 | 405992 | 45.40 | 0.007 |
| X₃ | 212.3 | 720865 | 80.62 | 0.003 |
| X₄ | −35.1 | 19679 | 2.20 | 0.235 |
| X₅ | 33.9 | 18363 | 2.05 | 0.247 |
| X₁ × X₂ | −33.5 | 17977 | 2.01 | 0.251 |
| X₁ × X₄ | 34.0 | 18493 | 2.07 | 0.246 |
| X₁ × X₆ | 50.1 | 40168 | 4.49 | 0.124 |
| X₂ × X₄ | 89.0 | 126744 | 14.17 | 0.033 |
| Curvature | | 237603 | 26.57 | 0.014 |
| Residual | | 8942 | | |
| Lack of fit | | 7435 | 0.62 | 0.668 |
| Pure error | | 11956 | | |

^{a}The results with P-values higher than 0.3 are not shown.
*R2 = 0.9839

### [Example 7] Steepest Ascent Method

To determine the optimal concentrations of the two ingredients selected by FFD, first, a steepest ascent method for detecting an approximate optimal concentration was carried out. In the determined concentration range, the higher the concentrations of both components, the higher the amount of production of colanic acid. Therefore, an approximate optimal concentration range for both components was determined by measuring the degree of bacterial growth and the production amount of colanic acid while increasing the concentrations of both components together. Referring to Table 4, as expected, as the concentrations of both components increased, the production amount of colanic acid increased and reached the maximum value in the 8^{th} experiment. Accordingly, it was confirmed that the optimal conditions were about 2.90 g/l for tryptone, and about 10.98 g/l for Na₂HPO₄.

TABLE 4
Experiment design by steepest ascent method and its result

| Run | X₂ | X₃ | CA (mg/l) | OD₆₀₀ |
|---|---|---|---|---|
| 1 | 1.50 | 6.78 | 1224.9 ± 11.4 | 1.52 ± 0.06 |
| 2 | 1.70 | 7.38 | 1326.7 ± 78.3 | 1.70 ± 0.01 |
| 3 | 1.90 | 7.98 | 1391.3 ± 179.4 | 1.85 ± 0.04 |
| 4 | 2.10 | 8.58 | 1593.3 ± 107.0 | 2.08 ± 0.08 |
| 5 | 2.30 | 9.18 | 1581.3 ± 69.9 | 2.23 ± 0.08 |
| 6 | 2.50 | 9.78 | 1691.0 ± 82.2 | 2.34 ± 0.07 |
| 7 | 2.70 | 10.38 | 1830.0 ± 65.6 | 2.49 ± 0.06 |
| 8 | 2.90 | 10.98 | 1887.4 ± 110.4 | 2.67 ± 0.10 |
| 9 | 3.10 | 11.58 | 1701.4 ± 53.7 | 2.78 ± 0.04 |

### [Example 8] Surface Response Method Using Central Composite Design (CCD)

Based on the result obtained by the steepest ascent method, CCD was performed before and after the optimal conditions for the approximate concentrations of tryptone and Na₂HPO₄.

Specifically, to determine the optimal concentrations of two ingredients for a culture medium (that is, tryptone and Na₂HPO₄) for maximum production of colanic acid, CCD was performed with five code values (−1.414, −1, 0, 1 and 1.414). The code values of two factors (tryptone and Na₂HPO₄) were calculated using the following equation:

$${x}_{i} = \frac{{X}_{i} - {X}_{0}}{Δ {X}_{i}} \qquad ( 1 )$$

A three-dimensional model was selected to simulate the optimal concentrations of tryptone and Na₂HPO₄ based on regression analysis. The equation of the three-dimensional model is as follows:

γ=b₀+Σb₁xᵢ+Σb₂xⱼ+Σb₃xᵢxⱼ+Σb₄xᵢ²+Σb₅xⱼ²+Σb₆xᵢ²xⱼ+Σb₇xᵢxᵢ² (2)

Here, xᵢ and xⱼ are code values of independent variables, and y is an expected response (colanic acid production amount). Various regression coefficients affecting the response (y), such as b₀, b₁, b₂, b₃, b₄, b₅ and b₆, represent an intercept (b0), linear coefficients (b1, b2), a 2-factor interaction coefficient (b3), secondary coefficients (b4, b5) and tertiary coefficients (b6, b7). Each coefficient of the 3D model for colanic acid production was obtained by regression analysis. Based on a fixed model, a response surface plot was made to find the optimal concentrations of tryptone and Na₂HPO₄ for production of colanic acid using Design-Expert 7.0 (Stat-Ease, Minneapolis, Minn., USA).

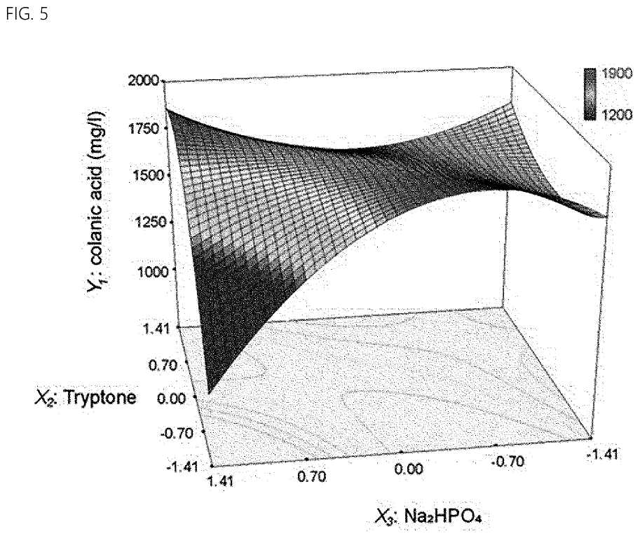

The result is shown in Table 5, and the regression analysis result is shown in Table 6. Based on these, the production amount of colanic acid according to the tryptone and Na₂HPO₄ concentrations was expressed in a 3D surface plot (FIG. 5). After that, a confirmation experiment was performed to find a point that leads to the maximum production amount of colanic acid.

TABLE 5
Experiment design by CCD and its result

| | Factor^{a} | | | |
|---|---|---|---|---|
| Run | x₂ | x₃ | CA (mg/l)^{b} | OD₆₀₀^{b} |
| 1 | −1 | −1 | 1771.3 ± 37.2 | 2.53 ± 0.01 |
| 2 | −1 | 1 | 1503.6 ± 48.9 | 2.57 ± 0.03 |
| 3 | 0 | −1.414 | 1649.7 ± 47.1 | 2.75 ± 0.01 |
| 4 | 0 | 0 | 1837.3 ± 65.0 | 2.63 ± 0.08 |
| 5 | 0 | 0 | 1704.2 ± 79.0 | 2.76 ± 0.02 |
| 6 | 0 | 0 | 1751.4 ± 96.0 | 2.72 ± 0.06 |
| 7 | −1.414 | 0 | 1873.4 ± 112.0 | 2.56 ± 0.11 |
| 8 | 1 | −1 | 1651.3 ± 221.6 | 2.33 ± 0.08 |
| 9 | 1.414 | 0 | 1639.5 ± 61.7 | 2.69 ± 0.09 |
| 10 | 0 | 0 | 1808.7 ± 110.8 | 2.71 ± 0.02 |
| 11 | 0 | 0 | 1840.2 ± 171.0 | 2.68 ± 0.13 |
| 12 | 1 | 1 | 1774.5 ± 101.0 | 2.62 ± 0.10 |
| 13 | 0 | 1.414 | 1788.6 ± 188.9 | 2.72 ± 0.01 |

^{a}x₂ = (X₂ − 2.9)/0.2; x₃ = (X₃ − 10.98)/0.6
^{b}Data were expressed as means ± standard deviations of triplicate experiments.

TABLE 6
Regression analysis for CCD result

| Source | Coefficient estimate | Mean square | F-value | p-value |
|---|---|---|---|---|
| Model | | 15961.07 | 4.71 | 0.054 |
| Intercept | 1795.22 | | | |
| X₂ | −82.68 | 27345.92 | 8.06 | 0.036 |
| X₃ | 49.10 | 9644.83 | 2.84 | 0.153 |
| X₂ × X₃ | 97.72 | 38198.51 | 11.26 | 0.020 |
| X₂² | −35.06 | 8551.89 | 2.52 | 0.173 |
| X₃² | −53.68 | 20042.24 | 5.91 | 0.059 |
| X₂² × X₃ | −85.26 | 14537.75 | 4.29 | 0.093 |
| X₂ × X₃² | 120.42 | 29000.23 | 8.55 | 0.033 |
| Residual | | 3391.46 | | |
| Lack of fit | | 7848.15 | 3.45 | 0.137 |
| Pure error | | 2277.28 | | |

*R² = 0.8682

### [Example 9] Confirmation of Optimal Medium

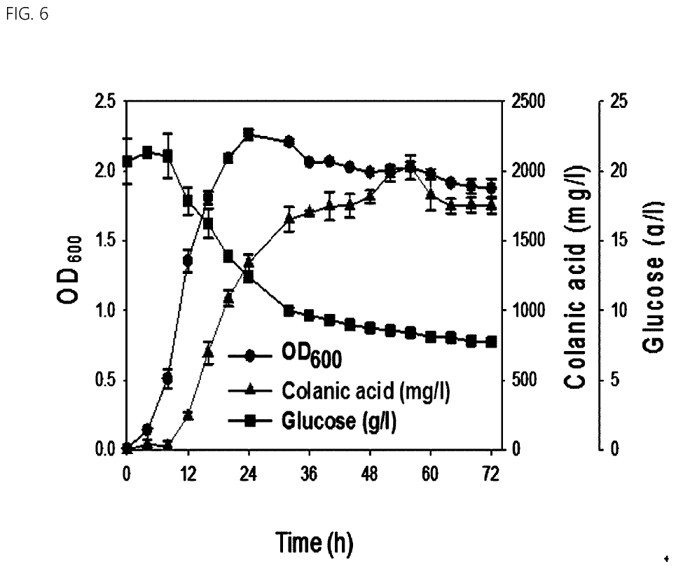

As a result of cultivation in a fermentation medium containing 20.00 g/l of glucose, 2.63 g/l of tryptone, 10.62 g/l of Na₂HPO₄, 3.00 g/l of KH₂PO₄, 0.50 g/l of NaCl, 0.24 g/l of MgSO₄, and 0.011 g/l of CaCl₂ at 25° C. and 200 rpm, as the optimal conditions, it was confirmed that the maximum colanic acid production amount is 2052.8 mg/l (FIG. 6). It was confirmed that the production amount is a value about 10-fold higher than that before optimization of a M9 medium.

1. A method of producing colanic acid, comprising:
preparing a mutant E. coli JM109 strain by removing the waaF gene from an E. coli JM109 strain; and
culturing the prepared mutant E. coli JM109 strain in a fermentation medium.

2. The method of claim 1, wherein the fermentation medium comprises glucose, tryptone and sodium phosphate (Na₂HPO₄).

3. The method of claim 2, wherein the fermentation medium further comprises sodium chloride (NaCl), magnesium sulfate (MgSO₄), calcium chloride (CaCl₂) and potassium phosphate (KH₂PO₄).

4. The method of claim 3, wherein the fermentation medium comprises 10 to 30 g/l of glucose, 7 to 15 g/l of sodium phosphate, 1 to 5 g/l of potassium phosphate, 0.1 to 1 g/l of sodium chloride, 1 to 5 g/l of tryptone, 0.1 to 0.5 g/l of magnesium sulfate and 0.005 to 0.02 g/l of calcium chloride.
